(19) United States
(12) Patent Application Publication
(10) Pub. No.: US 2003/0144473 A1
(43) Pub. Date: Jul. 31, 2003

(54) Peptides for the production of preparations for the diagnosis and therapy of autoimmun diseases

(76) Inventors: Michael Zeppezauer, Scheidt (DE); Arno Schonberger, Fonhold (DE); Ladislav Cebecauer, Piestany (SK)

Correspondence Address: GREENBERG TRAURIG, LLP, 21st Flr., 885 Third Avenue, New York, NY, 10022, US

(21) Appl. No.: 09/988,165

(22) Filed: Nov. 19, 2001

Related U.S. Application Data
(63) Continuation-in-part of application No. 07/946,180, filed on Sep. 16, 1992, now Pat. No. 6,369,203.

(51) Int. Cl. A61K 39/395; G01N 33/53; C07K 16/42; C07K 7/08

(52) U.S. Cl. 530/326; 514/14; 530/387.2; 435/7.1; 424/131.1

(57) ABSTRACT

Peptides are proposed with antigenic or immunogenic determinants, which result from autoantibodies in the body fluids of patients, who are suffering from autoimmun diseases, in particular diseases of the rheumatic group as systemic lupus erythematosus (SLE), rheumatoid arthritis or systemic sclerosis. In the case of the peptides it is preferably a question of the C terminus of bovine histone H1 with the sequence section 187-211 or corresponding human histon-H1-peptides of the sub-types H1.1, H1.2, H1.3, H1.4, H1.5 and H1.a and the N termini of histone H2B with the sequence sections 1-35 and 36-76, which are capable of cross reactions with the autoantibodies (anti-histone-antibodies). The invention furthermore provides ways of forming monoclonal antibodies and antiidiotypical antibodies, which are directed against autoantibodies. The diagnosis of autoimmun diseases is possible in accordance with the invention with a high degree of certainty and the monoclonal antibodies directed against the autoantibodies are suitable for the production of medicaments for the therapy of said diseases.

This application is a continuation of the pending U.S. patent application Ser. No. 07/946,180 filed In the United States Patent Office on Sep. 16, 1992 entitled “Peptides For The Production Of Preparations For The Diagnosis And The Therapy Of Systemic Lupus”.

The present invention relates to peptides with antigenic or immunogenic determinants, which may be recognized by autoantibodies in the body fluids of patients, who are suffering from an autoimmun disease, in particular diseases of the rheumatic group as systemic lupus erythematosus (SLE), rheumatoid arthritis or systemic sclerosis.

Autoimmun diseases in particular diseases of the “rheumatic group” are characterized by a large number of clinical phenomena and by a wide spectrum of autoantibodies. The latter are directed against various different components of normal cells. The said diseases include for instance systemic lupus erythematosus (SLE) which may occur spontaneously or may be induced by medicaments or drugs. In the case of SLE the occurence of autoantibodies is particularly frequent, which are directed against components of the cell nucleus (antinuclear antibodies, ANA's), these including inter alia double strand desoxyribonucleic acid (DS-DNA) and histone proteins, ribonucleic acid (RNA), complexes of DNA and histones as well as enzymes.

Histones consist of a number of classes of proteins, the so-called core histones H2A, H2B, H3 and H4, which are found in the nucleosomes, and the linker histones H1 and H5, to which linking functions are attributed in the formation of chromatin. To proteins of all these classes, or fragments derived thereof, additional functions have been attributed, notably hormonal or hormone-like functions, cytokine-like functions and defense functions against foreign cells, i.e. tumor cells, bacteria and fungi identifying the histones as components of the innate immune defense. Many attempts have been made to correlate the frequency of autoantibodies, which are directed against special antigens, with certain rheumatic syndromes.

It has been discovered that regarding autoimmun patients, particularly SLE-patients, autoantibodies (AHA's, anti-histone autoantibodies) occur more frequently against histones. However, it is also known that AHA's which occur in SLE are in addition associated with other immunological disorders, as well, e.g. with rheumatoid arthritis and systemic sclerosis. Normally the “enzyme linked immuno-sorbent assay (ELISA) is utilized for determination, in the case of which the sera of patients and of healthy control subjects are tested on purified cell components (i.e. antigens). Pure histone is inter alia employed as an antigen for the testing of SLE sera.

Furthermore additionally synthetic peptides or those produced by the degradation of natural histones are used, which consist of sequence parts of the said histones.

In this respect it has been seen that in the case of use of the individual histones and histone peptides:

(i) the frequency of a positive reaction in an ELISA is not greater than 50% and that

(ii) the frequency of a positive reaction in the case of patient sera related to other rheumatic diseases is large (false positive results).

Thus recently a study concerning the predictive value of recognition of AHA's of autoimmun patients, in particular SLE-patients (by means of the LE cell test, SmeenK et at., Scand. J. Rheumatology. Suppl. 56, 78-92, 1985) came to the following conclusion; although 95% of SLE patients were positive in the LE test, in fact the chances that a patient with a positive LE test has SLE are only 27%.

Therefore it is an abject of the invention to improve the predictive value of diagnostic tests for autoimmun diseases, in particular for SLE or rheumatoid arthritis, or systemic sclerosis, that is to say if the percentage of true-positive results as related to false positive ones could be increased.

Furthermore to receive a therapeutic method of the invention it could be valuable if monoclonal antibodies and monoclonal antiidiotypical antibodies, which are specific in the very same manner as the antibodies of autoimmun patients, in particular SLE-patients, or rheumatoid arthritis patients or systemic sclerosis patients, and if monoclonal antibodies and monoclonal antiidiotypical antibodies could be produced, which are directed against these monoclonal antibodies or, respectively, the autoantibodies of said autoimmun patients.

Furthermore to receive a therapeutic method of the invention it would be valuable to prevent the formation of autoantibodies or reduce their concentration in the body in order to prevent or delay the onset and/or the development of these syndromes in which the formation of autoantibodies plays a role in pathogenesis and/or progression.

In order to achieve these and/or other objects appearing from the present specification and claims in the present invention a peptide with antigenic or immunogenic determinants, which is recognized by autoantibodies, more particularly in the body liquids of a patient suffering from an autoimmun disease, in particular diseases of the rheumatic group as systemic lupus erythematosus (SLE), rheumatoid arthritis and systemic sclerosis, is characterized in that at least one of the following peptides or their effective parts (at least an amino acid sequence of at least 8 amino acids) are selected from the group consisting of

| | |
|---|---|
| (1₁) | KPKAA KPKAA KPKAA KPKKA APKKK, |
| (1₂) | KPKAA KARUT KPKTA KPKKA APKKK |
| (1₃) | AAKAV KPKAA KPKVV KPKKA APKKK |
| (1₄) | KPKAA KPKSG KPKVT KAKKA APKKK |
| (1₅) | KPKAA KPKTA KPKAA KPKAA AAKKK |
| (1₆) | KPKAA KPKAA KPKAA KAKKA AAKKK |
| (1₇) | KPKAA KPKAA KPKAA KP KAKKA AAKKA |
| (2) | PEPAK SAPAP KKGSK KAVTK AQKKD GKKRK RSEKE, |
| | and |
| (3) | SYSVY VYKVL KQVHP DTGIS SKAMG IMNSF VNDIF |
| | ERIAGE. |

The above mentioned amino acid sequences are expressed in single letter codes.

The effective parts of the peptides have hormonal or hormone-like functions and/or cytokine-like functions.

Further advantageous developments and convenient forms of the invention will be gathered from the features of the further claims and the following description.

The following natural and synthetic peptides were tested (expressed in one letter codes as follows:

Histon-H1-Peptide (bovine peptides)
H1-N-Terminus: 3-29
A P A A P A A A P P A E K T P V K K K A A K K
P A G A
H1: 55-75
R S G V S L A A L K K A L A A A G Y D V E
H1: 97-116
T K G T G A S G S F K L N K K A A S G E
H1: 76-116
K N N S K I K L G L K S L V S K G T L V E T K G
T G A S G S F K L N K K A A S G E
H1: 66-116
A L A A A G Y D V E K N N S R I K L G L K S L V
S K G T L V E T K G T G A S G S F K L N K K A A
S G E
H1: 55-166
R S G V S L A A L K K A L A A A G Y D V E K N N S
R I K L G L K S L V S K G T L V E T K G T G A S Q
S F K L N : K K A A S G E
H1-C-Terminus: 187-211
K P K A A K P K A A K P K A A K P K K A A P K K K
Histon H2B-Peptide (bovine or human peptide)
H2B: 1-35
P E P A K S A P A P K K G S K K A V T K A Q K K
D G K K R K R S E K E
H2B: 36-76
S Y S V Y V Y K V L K Q V H P D T G I S S K A M G
I M N S F V N D I F E R I A G E
H2: 77-93
A S R L A H Y N K R S T I T S R E
H2B: 94-105
I Q T A V R L L L P G E
H2B: 106-113
L A K H A V S E
H2B: 133-124
G T K A V T K Y T S S R
H2B: N-Term. 1-21
P E P A K S A P A P K K G S K K A V T K A
H2B N-Term: 4-11
A K S A P A P K
Histon H2A-Peptide
H2A-N-Terminus
S G R G K Q G G K A R A K A K T R S S R A G
Histonsequenzen: (bovine or human peptied)
H2A:
S G R G K Q G G K A R A K A K T R S S R A G L Q F
P V G R V H R L L R K G N Y A E R V G A G A P V Y
L A A V L E Y L T A E L L E L A G N A A R D N K K
T R I I P R H L Q L A I R N D E E L N K L L G K V
T I A Q G G V L P N I Q A V L L P K K T E S H H K
A K G K.

The inventors are aware that the amino acid sequences of histones from different animal species are only in part known, today. However, it has frequently been observed that the amino acid sequences of the individual subtypes of H1, H2A, H2B, H3 and H4 are closely similar, even in more distantly related animal species. It is generally believed that these findings reflect a strict evolutionary conservation of the characteristic amino acid sequences of individual histone subtypes.

The inventors are also aware that the amino acid sequences of the C terminal parts of the histone H1 subtypes of human and as far as they are known of consensus sequences, i.e. bovine and other mammals are very similar. They are composed of homologous sequence patterns (boxes) of the type K P K A A, K P K K A, K A K K A or boxes derived from them by exchange of one or two amino acids.

The final box is A P K K K or A A K K K.

The following table depicts these C terminal sequences of human histone H1 subtypes 1.1, 1.2, 1.3, 1.4, 1.5 and 1.a:

Histon-H1-Peptide (human peptide)
H 1.1: 191-215
KPKAA KARUT KPKTA KFKKA APKKK
H 1.2: 193-218
AAKAV KPKAA KPKVV KPKKA APKKK
H 1.3: 195-220
KPKAA KPKSG KPKVT KAKKA APKKK
H 1.4: 191-218
KPKAA KPKTA KPKAA KPKAA AAKKK
H 1.5: 195-225
KPKAA KPKAA KPKAA KAKKA AAKKK
H 1.a; 196-222
KPKAA KPKAA KPKAA KP KAKKA AAKKA

From the peptides 1₁ til 1₇ smaller peptides may be selected which contain at least eight amino acids and at least one consensus sequence (depicted as boxes of five amino acids) whereby the C terminal is always A x K K K (x=A or P).

The inventors therefore expect that the immunological properties of the peptides disclosed herein will also be observed with the homologous amino acid sequences of histones from different animal species, including human species, which will be revealed in the near future. Therefore the present invention also comprises the immunological properties as disclosed herein of peptides corresponding to homologous histone sequences from different animal and human species which will be revealed in the coming years and which will be readily recognized by the skilled in the art who compares the new histone sequences with the peptides disclosed herein.

By means of ELISA the epitopes of the autoantibodies of 112 rheumatic and SLE sera were charted with H1, H2B and H2A peptides 80% of the SLE sera and 66% of all sera reacted positively both to the C terminus of H1 and also to the N terminus of H2B. The combination of the two regions is therefore to be regarded as a marker sequence and hence as a distinguishing criterion for SLE patients. Both the structural data concerning these regions as well as the antigenity calculations the homologous epitopes of the patent's own antibodies produced in vivo and in vitro underline the dominant antigenic character of the N terminus of H2B and of the C terminus of H1.

For the ELISA (enzyme linked immuno-sorbent assay) F16 modules of the Nunc Company were utilized with a special highly active surface. Dependent on the purpose of the test eltzher the antibody to be tested (in a direct ELISA or sandwich test) or the antigen (in an indirect ELISA) were bound to the surface of the microtitration plate. The antigens were dissolved with a concentration 50 μg/ml in a 0.05 M carbonate buffer, pH 9.7. Antibody solutions, supernatant liquid from cells and urine samples were diluted 1 to 3 in the same buffer and in each case 100 μl were pipetted onto the microtitration plate. Linking took place for 24 hours at 4° C. After emptying the dishes on the following day reactive groups of the microtitration plate were blocked at 36° C. with 250 μl of blocking solution per dish. For this different blocking solutions were employed: 0.5% (w/v) gelatins in PBS/azide; 1% (w/v) BSA in PBS/azide; 5% (w/v) BSA in PBS/azide; 10% (v/v) equine serum in PBS/azide. The additon was then made of 100 μl of cell culture supernatant liquid (primary antibodies) or, respectively, the 1 to 250 diluted sera with incubation for one hour at room temperature in the dark. After rinsing the microtitration plate once with Tween solution (consisting of 0.1% (v/v) Tween 20 and 150 mM NaCl) 100 μl of conjugate (0.3% (v/v) rabbit anti-(mouse-igG)IgG-HRP or, respectively, rabbit-anti(human-IgG)IgG-HRP were applied thereto by pipetting and incubated at room temperature for one hour. Unbound antibodies were removed by rinsing five times with Tween solution. After the addition of 100 Ml of Mcllvanie buffer (116 mM Na₂HPO₄+2H₂O, 42 mM citric acid, pH 5.6, including 1.5 mM of orthophenylenediamine and 0,9 mM of H₂O₂), the horseradish peroxidase, coupled with the rabbit or, respectively, ovine antobodies, completed the color reaction in the dark, it being arrested with 100 μl of 2 M H₂SO₄. After matching against the blind sample, the extinction of the different dishes was ascertained, photometrically at 490 nm with the aid of a Minireader II and a print out was made of the values obtained.

The 122 SLE sera were tested for autoantibodies in an ELISA which indicated that the N terminal range (1-35) of H2B and the C terminal range (187-211) of H1 represent preferred epitopes of SLE autoantibodies. Furthermore the dilution rate of 1 to 250 was found to be more particularly suitable for detection of a wide spectrum of high and low titer sera in an ELISA. In this respect the inventors turned their main attention to the IgG-autoantibodies.

The results were evaluated as follows: an ELISA on a patient was only rated as positive if both extinctions>0.2 (cut off=0.2 from dummy measurements and correction factor in the case of stray values>0.2) and distinctly higher than the values in comparison with all other peptides.

Of the 122 sera 68% were positive with respect to the peptide combination. The 122 sera were composed of 80 SLE and 42 rheumatic sera. Of the 80 lupus patients 80% were H1-CT and E1 positive, whereas of the 42 rheumatic patients 45% were still H1-CT and E1 positive. Therefore the N terminal ranges of H2B (1-31) and the C terminal range of H1 (187-211) constituted the main antigenic determinants detected of autoantibodies of the lupus patients. The combination of thes4e two peptides may therefore function as a distinguishing criterion for the classification of SLE patients and separating them from rheumatic patients.

In order to produce the monoclonal antibodies (anti-histone-antibodies), which are directed against the autoantibodies in the body liquids of SLE patients, the procedure adopted in the invention was as follows (schedule I):

(1) Analysis of the histone sequences (mathematical) model).

(2) Prediction of the antigenic ranges.

(3) Synthesis of peptides in accordance with the antigenic ranges. The peptides are partly produced in a free condition and partly bound on a carrier (TentaGel).

(4a) lmmunization of animals (mice) with synthetic peptides In accordance with (3); the peptides must be used bound to a carrier (as for instance on a TentaGel)

(4b) Immunization of spleen cells in vitro with synthetic peptides in accordance with (3). In this case free or carrier-bound peptides may be employed.

(5) Isolation of the spleen cells and fusion with cancer cells to give hybridoma cells; selection of individual (positive) clones.

(6) Isolation of the exuded anti-histone-antibodies (A-HA).

(7) Investigation of specificity and activity of the synthetic AHA's using synthetic peptides in accordance with (3) as antigens by means of an ELISA.

In order to produce the antiidiotypical antibodies in accordance with the invention the procedure was as follows in accordance wit the invention (schedule II):

(1.1) Selection of the antigen:

The antigen is for instance an epitope directed against histone peptides H1 (187-211) and H2B (1-35), on the autoantibody in the serum of SLE patients or

(1.2) The corresponding epitope on the monoclonal antibodies, which were produced against this peptide/peptide combination.

(2) Production of the antigen(s).

(2.1) The antibody fraction of the SLE serum is enriched using a conventional method.

(2.2.1) Those autoantibodies are selectively removed from the enriched antibody fraction of the SLE serum by affinity chromatography, which have the epitopes as defined in (1). For this purpose the peptides defined in 81) are bound using suitable methods on suitable carrier materials chemically or adsorptively).

As an alternative it is possible as well for the peptides to be synthesized on suitable carrier materials, as for instance TentaGels. It is consequently possible to firstly pass the enriched antibody fraction of the SLE serum through a column with carrier H1 (187-211)-conjugate, to wash it and then to elute the autoantibodies bound on the conjugate using a suitable method. This autoantibody fraction is then passed in a second step through a column with a carrier-H2B (1-35)-conjugate. The double specific or cross specific autoantibodies of interest are then retained and after washing of the column using a suitable method may be eluted. It is furthermore possible to change over the order of affinity steps as well, that is to say firstly to use the carrier-H2B (1-35) and then the carrier-H1C.

(2.2.2) The monoclonal antibodies, which in accordance with (1.2) possess the double specific epitope, are isolated in accordance with schedule I (6) and then purified.

(3) Immunization methods

(3.1) In vivo immunization

The autoantibodies produced in accordance with (2), or monoclonal antibodies are used in the conventional manner for immunization. They may be freely employed in combination with suitable adjuvants or coupled with a suitable carrier, as for instance a Tantagel.

(3.2) In vitro immunization

The antibodies produced in accordance with (2) may be employed as well in order to immnunize spleen cells of suitable experimental animals in vitro using conventional methods.

(4) Isolation of the spleen cells producing antiidiotypical antibodies and fusion with suitable cancer cells to give hybridoma cells.

(5) Selection and culture of individual clones.

(6) Isolation and purification of the monoclonal antiidiotypical antibodies.

It would also be possible not to use step (3) but rather to isolate β-lymphocytes from the blood of SLE patients (or of animals with autoimmune diseases), to fuse them with tumor cells and to isolate those clones from the resulting hybridoma cells which have the specificity noted in (1). The identification of these clones is performed by means of conventional tests, as for instance ELISA, using the peptide/peptide combinations in accordance with the invention.

It is clear that the determination of the concentration of the autoantibodies (anti-histone-antibodies) of SLE patients is not limited to ELISA-type-methods.

The AHA concentration may furthermore be determined by radioimmune assay (RIA) using radioactive marked N terminal peptides of H2B and C terminal peptid3s of H1 or by means of a fluorescence-immuno assay with N terminal peptides, marked to fluoresce, of H2B and C terminal peptides of H1. It will be clear to the man in the art that the detection and ascertainment of concentration for AHA may be performed in other body liquids and components thereof, as for instance urine, besides sera.

It has been found in accordance with the invention that antigenic determinants of the histones H1 and H2 may be characterized both by means of synthetically produced monoclonal antibodies end also by means of human pathogenic autoantibodies. In order to improve the autogenic properties of the very conservative and weakly immunogenic histones, purified classes of histones or selected synthetic peptides are coupled with different carriers.

In vivo immunization with glutaraldehyde cross linked histone complexes lead to an IgM antibody (1/A8/B1) which is directed against conformation antigens. In vivo immunization with histone H1 coupled to Eupergit C led to three further monoclonal IgM antibodies: 1/H4/C3 (IgG₂ₐ, 1/H4/C6 (IgG₂ₐ) and 1/H4/C10 (IgG₂ₐ), all three having a kappa specificity of the light chain. The epitope of the three monoclonal antibodies was In the C terminus of H1 (187-211). The cross reaction of the antibodies with the T terminus of H2B (22-35) is to be attributed to the sequence and charge homology of the two terminal histone ranges. Two N terminal peptides from H2B, coupled with Eupergit, were employed for in vivo immunization.

As antigens free histones, free peptides and peptides coupled with carriers were used. In vitro immunization with free histone H1 led to an IgG₂ₐantibody with a kappa chain, whose epitope is also the C terminus of H1.

In accordance with the invention it was possible to use TentaGels as a new synthetic carrier material for successful in vitro immunization. TentaGels constitute a new class of grafted copolymeric particles, whose polystyrene nucleus is surrounded by “marginal bush-like” polyoxyethylene tentacles. These carriers may be employed in a “single step method” after peptide synthesis immediately for in vitro immunization. TentaGels are characterized by a very high biocompatibility, chemical inertness, improved hydrophilio properties and last but not least by optimum exposure of uniform haptenic structures for contact with immune-competent cells.

The monoclonal antibodies produced are employed both in different immunological test systems, such as immunodiffusion, hemagglutination, dot blot and various ELISA systems as well as, air coupling with fluorescing isothyocyanate (FITC) and horseradish oxidase (HRP) for the performance of continuous flow cytometry and in the Western blot test.

The invention also comprises the use of the peptides of the invention in the therapy of immunological disorders, in particular of SLE, rheumatoid arthritis and sclerodermia. In the therapeutical methods of the invention a pharmaceutical composition which comprises a therapeutically effective amount of at least one peptide with an amino acid sequence as disclosed herein in SEQ. ID. NO. 1, 2 or 3 is administered to a patient. A therapeutically effective amount of a peptide is an amount which upon single or repeated administration to a patient does alleviate an inflammation or reduce any symptom of the aforementioned disorders. The pharmaceutical compositions of a first embodiment of the invention comprise at least one lyophilised peptide of SEQ. ID: NO. 1 to 3 in dry form, which can be readily dissolved, e.g. in phosphate-buffered saline (PBS), aqua ad injectabilia, Ringer's solution or the like, prior to use. The pharmaceutical compositions may also comprise pharmaceutically acceptable carriers. The pharmaceutical preparations are preferably administered by parenteral injection, renal perfusion, or by oral application. The pharmaceutical compositions of the invention are in specialised embodiments adapted to various oral or topical applications to a patient. The skilled in the art readily prepares the suitable compositions. The pharmaceutically effective amount of a single dose of at least one peptide of the invention depends on the age and site of the patient, on the route of administration, and the severeness of the symptoms. Without any restriction, a therapeutically effective amount of a peptide may range from 0.1 to several hundred milligrams. In an advantageous embodiment, the pharmaceutical composition comprises the peptides of SEQ. ID. NO. 1 and SEQ. ID. NO. 2 in equimolar amounts.

According to the invention peptides re proposed with antigenic or immunogenic determinants, which result from autoantibodies in the body fluids of patients, who are suffering from autoimmun diseases, in particular diseases of the rheumatic group as systemic lupus erythematosus (SLE), rheumatoid arthritis or systemic sclerosis. In the case of the peptides it is preferably a question of the C terminus of bovine histone H1 with the sequence section 187-211 or corresponding human histon-H1-peptides of the sub-types H1.1, H1.2, H1.3, H1.4, H1.5 and H1.a and the N termini of histone H2B with the sequence sections 1-35 and 36-76, which are capable of cross reactions with the autoantibodies (anti-histone-antibodies). The invention furthermore provides ways of forming monoclonal antibodies and antiidiotypical antibodies, which are directed against autoantibodies. The diagnosis of autoimmun diseases is possible In accordance with the invention with a high degree of certainty and the monoclonal antibodies directed against the autoantibodies are suitable for the production of medicaments for the therapy of said diseases.

What is claimed is:

1. At least one of the following peptides or their effective parts with hormonal or hormone-like function and/or cytokine-like function selected from the group consisting of

| | |
|---|---|
| (1₁) | KPKAA KPKAA KPKAA KPKKA APKKK |
| (1₂) | KPKAA KARVT KPKTA KPKKA APKKK |
| (1₃) | AAKAV KPKAA KPKVV KPKKA APKKK |
| (1₄) | KPKAA KPKSG KPKVT KAKKA APKKK |
| (1₅) | KPKAA KPKTA KPKAA KPKAA AAKKK |
| (1₆) | KPKAA KPKAA KPKAA KAKKA AAKKK |
| (1₇) | KPKAA KPKAA KPKAA KP KAKKA AAKKA |
| (2) | PEPAK SAPAP KKGSK KAVTK AQKKD GKKRK |
| | RSEKE, and |
| (3) | SYSVY VYKVL KQVHP DTGIS SKAMG IMNSF |
| | VNDIF ERIAGE |

is used in the diagnosis and/or therapy of autoimmun diseases, in particular diseases of the rheumatic group as systemic lupus erythematosus, rheumatoid arthritis or systemic sclerosis.

2. Effective part of a peptide (11 to 17) according to claim 1 containing at least eight amino acids and/or including at least one consensus sequence depicted as boxes of five amino acids whereby the C terminal is always A x K K K (x=A or P).

3. A method for improving diagnosis of autoimmun diseases, in particular diseases of the rheumatic group a systemic lupus erythematosus (SLE), rheumatoid arthritis or systemic sclerosis comprising a first step, wherein a first peptide or their effective part according to claim 1 is used as a first antigen and a tissue sample taken from the body of a patient is brought into contact with said first antigen and specific binding of an antibody comprised in said tissue sample to said first peptide is detected to receive an antibody specifically bound to an antigen, and at least a second step, wherein a second peptide or their effective part according to claim 1 is used as a second antigen and is brought into contact with said tissue sample and specific binding of an antibody comprised in said tissue sample to said second peptide is detected by said method for the detection of an antibody specifically bound to an antigen.

4. A method according to claim 3 comprising an intermediate step between said first step and said second step, wherein said antibody, which in said first step has specifically bound to said first peptide, is eluted from said first peptide or its affective part for eluting of a bound antibody, and said eluted antibody is used in said second step.

5. A method according to claim 3 or 4, wherein said tissue sample is a serum sample, a blood sample, a sputum sample, a liquor sample, a urine sample or a tear sample.

6. A method according to claim 3 or 4 using a peptide or its effective part according to claim 1 selected from the group (1₁) to (1₇) as said first peptide and using the peptide or its effective part according to claim 1 mentioned with (2) as said second peptide.

7. A method according to claim 3 or 4 using the peptide or its effective part according to claim 1 mentioned with (2) as said first peptide and using the peptide or its effective part according to claim 1 selected from the group (1₁) to (1₇) as said second peptide.

8. A method for therapy of autoimmun diseases, in particular systemic lupus erythematosus (SLE), rheumatoid arthritis or systemic sclerosis, comprising administering to a patient a pharmaceutical composition comprising a therapeutically active amount of at least two peptides or their effective parts according to claim 1.

9. A method according to claim 8, wherein said pharmaceutical composition is an injectable solution and is administered by an injection.

10. A method for the production of the antiidiotypic antibody, which specifically binds to the antigen-binding site of a monoclonal antibody, said monoclonal antibody specifically binding both a peptide or its effective part having an amino acid sequence selected from the group consisting of (1₁) to (1₇) of claim 1 and to a peptide or its effective part (2), comprising at least one step of a selection for a hybridoma clone, wherein said monocional antibody is used as an antigen.

11. A method for improving the diagnosis of an autoimmun disease, in particular diseases of the rheumatic group as systemic lupus erythematosus (SLE), rheumatoid arthritis or systemic sclerosis, comprising a step, wherein a tissue sample taken from the body of a patient is brought into contact with an antiidiotypic antibody according to claim 9 and specific binding of an antibody comprised in said tissue sample to said antiidiotypic antibody is detected to receive an antiidiotypic antibody which has specifically bound to an antibody.

12. A method according to claim 11, wherein said tissue sample is a serum sample, a blood sample, a sputum sample, a liquor sample, a urine sample or a tear sample.

13. A method for therapy of autoimmun diseases, in particular diseases of the rheumatic group as systemic lupus erythematosus (SLE), rheumatoid arthritis or systemic sclerosis comprising administering to a patient a pharmaceutical composition comprising a therapeutically active amount of an antiidiotypic antibody according to claim 9.

14. A method according to claim 13, wherein said pharmaceutical composition is an injectable solution and is administered by an injection.
